(19) United States
(12) Patent Application Publication
(10) Pub. No.: US 2011/0092913 A1
(43) Pub. Date: Apr. 21, 2011

(54) SAFETY SYRINGE WITH A NEEDLE RETRACTION DEVICE

(76) Inventor: CHEN-YI FANG, Taichung City (TW)

(21) Appl. No.: 12/976,563

(22) Filed: Dec. 22, 2010

Related U.S. Application Data
(63) Continuation-in-part of application No. 12/436,061, filed on May 5, 2009.

(51) Int. Cl. A61M 5/32

(52) U.S. Cl. 604/195

(57) ABSTRACT

A safety syringe with a needle retraction device includes a syringe cylinder, a push rod inserted in the syringe cylinder to push the medicinal liquid, a seat disposed at a front end of the syringe cylinder to receive a syringe needle, wherein the push rod includes a pushing knob with a through bore mounted at a top end thereof; and the seat includes a connection unit provided at a rear end thereof, the pushing knob allows to be pushed into the connection unit, because the pushing knob is pressed to reduce its outer diameter by using the through bore, thereby prevent injuring medicinal personals and obtaining safe protection.

The present invention is a CIP of application Ser. No. 12/436,061, filed May 5, 2009, the entire contents of which are hereby incorporated by reference.

### BACKGROUND OF THE INVENTION

1. Field of the Invention

The present invention has arisen to mitigate and/or obviate the afore-described disadvantages.

2. Description of the Prior Art

A conventional syringe has to be recycled by professional personnel after use, however, a syringe needle will probably injure the person who removes it off the syringe cylinder.

### SUMMARY OF THE INVENTION

The primary object of the present invention is to provide a safety syringe with a needle retraction device which comprises:

a syringe cylinder, a push rod inserted in the syringe cylinder to push the medicinal liquid, a seat disposed at a front end of the syringe cylinder to receive a syringe needle, wherein the push rod includes a pushing knob with a through bore mounted at a top end thereof; and the seat includes a connection unit provided at a rear end thereof, the pushing knob allows to be pushed into the connection unit, because the pushing knob is pressed to reduce its outer diameter by using the through bore, thereby prevent injuring medicinal personals and obtaining safe protection.

### BRIEF DESCRIPTION OF THE DRAWINGS

FIG. 1 is a perspective view of a safety syringe with a needle retraction device in accordance with a preferred embodiment of the present invention;

FIG. 1A is a perspective view of a pushing knob of a push rod of FIG. 1;

FIG. 2 is a cross sectional view illustrating the assembly of the safety syringe with a needle retraction in accordance with the preferred embodiment of the present invention;

FIG. 3 is a cross sectional view illustrating the push rob being pushed into a connection unit of a seat;

FIG. 4 is a cross sectional view illustrating the push rod being pulled backward to actuate the seat and a syringe needle to move into a syringe cylinder;

FIG. 5 is a cross sectional view illustrating a neck portion being twisted to separate apart from the push rod so that the seat and the syringe needle leave in the syringe cylinder.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

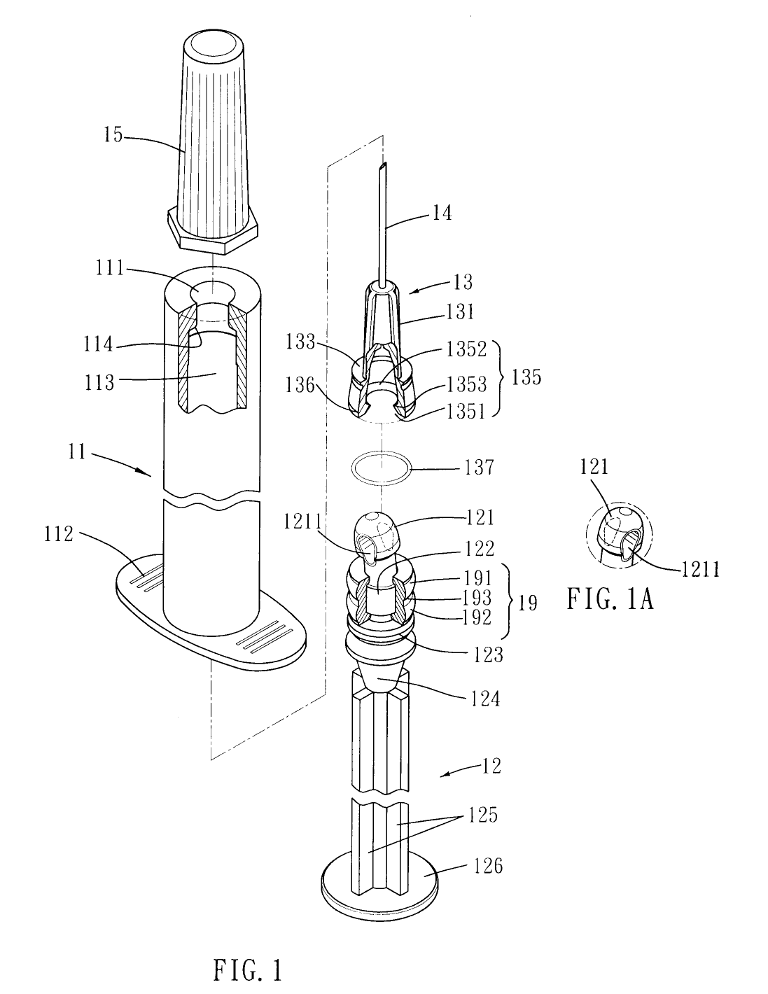

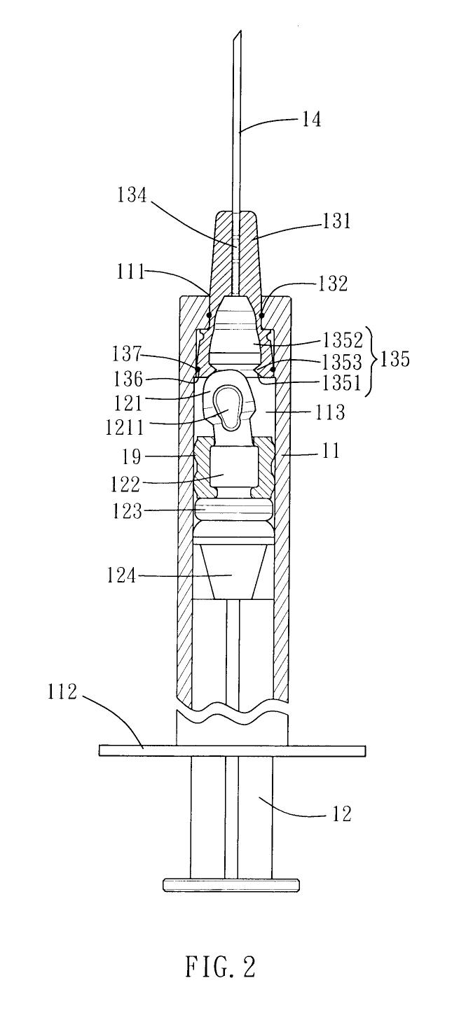

Referring to FIGS. 1 and 2, a safety syringe with a needle retraction device in accordance with the present invention comprises: a syringe cylinder 11, a push rod 12, a seat 13, a syringe needle 14, and a protective sleeve 15 for covering the syringe needle 14; wherein

the syringe cylinder 11 is formed in a cylinder shape, at a center of a front end of the syringe cylinder 11 is provided with a receiving hole 111 with a diameter, and at a bottom of a rear end thereof is disposed an enlarged flange 112 for holding the syringe cylinder 11 with user's thumb, the syringe cylinder 11 includes a receiving chamber 113 formed therein, and between an interior at a front end of the receiving chamber 113 and the receiving hole 111 is defined with a stop portion 114 with a reduced diameter;

the push rod 12 includes a hollow and raised pushing knob 121 with a predetermined diameter which is smaller than an inner diameter of the receiving chamber 113 of the syringe cylinder 11, and the pushing knob 121 includes a through bore 1211 arranged therein and communicating therewith, the through bore 1211 is composed of a laterally-oriented perforation and two openings disposed at both ends of the perforation, the through bore 1211 includes an upper diameter which is gradually enlarged toward an upper end of the pushing knob 121 and includes a lower diameter which is gradually reduced toward a lower end of the pushing knob 121, and the pushing knob 121 further includes a stem 122 extending downward from a bottom thereof, the stem 122 includes a combining portion 19 fitted thereon and having a first protrusion 191 and a second protrusion 192 which are both arranged on the combining portion 19 to correspond to the receiving chamber 113 of the syringe cylinder 11, and having a groove 193 defined between the first and the second protrusions 191 and 192, the stem 122 further includes an engaging rim 123 fixed at a lower side thereof, such that the receiving chamber 113 is tightly closed to prevent medicinal liquid from leak; besides, the combining portion 19 having the first protrusion 191, the second protrusion 192, and the groove 193 can be used to push the push rod 12 back and forth easily; the engaging rim 123 includes a neck portion 124 mounted at a lower side thereof and having a reduced diameter so as to rotate the push rod 12 after injection to leave a predetermined upper end of the neck portion 124 in the syringe cylinder 11, and a lower side of the neck portion 124 is connected with a plurality of longitudinal ribs 125 which have a circular tab 126 extendedly coupled on distal ends thereof to be pushed by the user's thumb; the pushing knob 121 of the push rod 12 is inclined at a predetermined angle in relation to the stem 122;

the seat 13 includes a fitting post 131 with a reduced diameter, at a suitable position of the fitting post 131 is engaged a peripheral projection 132 to prevent the seat 13 from returning to the receiving hole 111 of the syringe cylinder 11 during injection and to move the fitting post 131 out of the receiving hole 111, at that time, a positioning portion 133 of the fitting post 131 is biased against the stop portion 114 of the syringe cylinder 11, the fitting post 131 includes a passage 134 formed therein to receive the syringe needle 14, and the passage 134 includes a connection unit 135 provided at a rear end thereof, the connection unit 135 includes a slot 136 arranged at a lower side of an outer surface thereof to receive an anti-leak ring 137, such that the connection unit 135, the peripheral projection 132 of the fitting post 131, the combining portion 19 of the push rod 12, and the engaging rim 123 can completely close the receiving chamber 113 to prevent the medicinal liquid from leak, and the medicinal liquid filled in the receiving chamber 113 of the syringe cylinder 11 is injected into a patient's body through a communicating channel of the receiving chamber 113, the connection unit 135, the passage 134, and the syringe needle 14; at an opening end of the connection unit 135 is mounted a first diametered segment 1351, a diameter of which is larger than an outer diameter of the pushing knob 121 of the push rod 12, and a second diametered segment 1352 with a smaller diameter is formed at an upper side of the connection unit 135 to communicate with the passage 134 and to correspond to the outer diameter of the pushing knob 121 of the push rod 12, between the first and the second segments 1351 and 1352 is defined with a reduced segment 1353 with the smallest diameter in the connection unit 135, and an inner diameter of the reduced segment 1353 is in response to a diameter at a connecting portion of the pushing knob 121 and the stem 122.

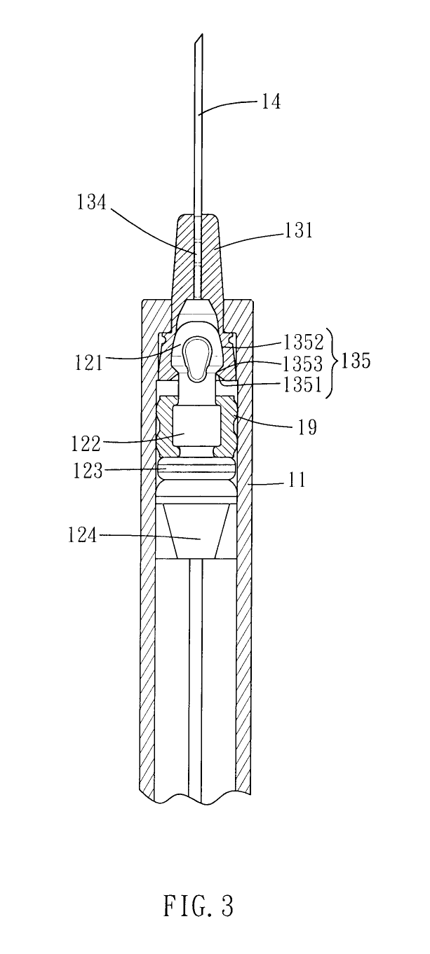

Referring to FIG. 3, as the medicinal liquid is nearly injected into the patient's body and the push rod 12 is continuously pushed forward, the pushing knob 121 contacts with the first diametered segment 1351 of the connection unit 135 of the seat 13 so that the pushing knob 121 is guided by the first diametered segment 1351 to straighten the size variable through bore 1211, and then the pushing knob 121 is further moved forward to the second diametered segment 1352. Although the diameter of the second diametered segment 1352 is smaller than that of the first diametered segment 1351, because the pushing knob 121 is flexible enough to be compressed to enter into the second diametered segment 1352 and then to return its original size to be positioned in the reduced segment 1353 of the connection unit 135. Furthermore, the push rod 12 and the seat 13 are tightly coupled together to inject entire medicinal liquid into the patient's body without any remains. In other words, the safety syringe with a needle retraction device of the present invention can be used to overcome the disadvantage of the conventional syringe, for example, the medicinal liquid is remained in the groove of the receiving chamber but not be injected into the patient's body completely.

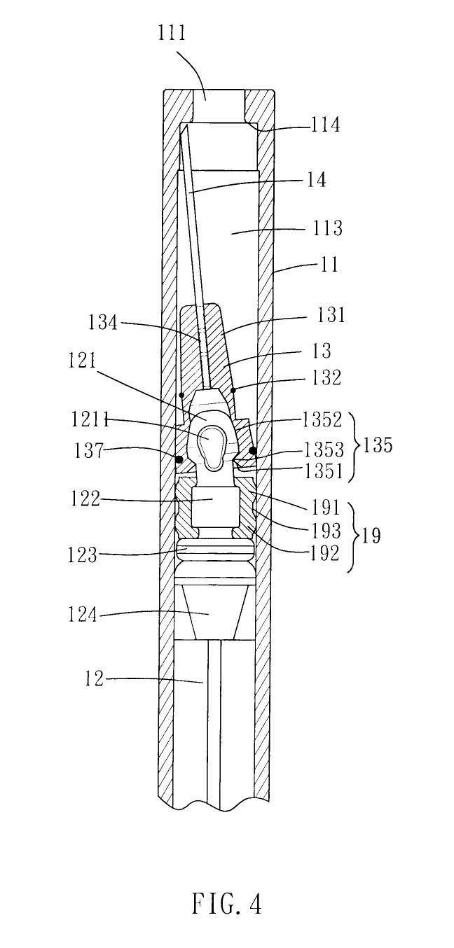

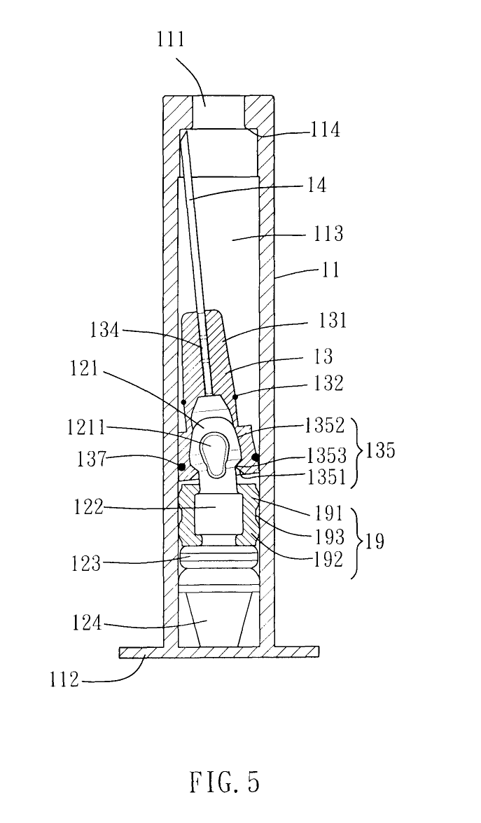

After injection, the push rod 12 is pulled backward to actuate the seat 13 and the syringe needle 14 to move into the receiving chamber 113 of the syringe cylinder 11. Due to the pushing knob 121 of the push rod 12 is inclined at the predetermined angle relative to the stem 122, the seat 13 and the syringe needle 14 also become inclined in the receiving chamber 113 without being pushed outward again as shown in FIG. 4, thus obtaining safe protection. When the neck portion 124 of the push rod 12 moves back to the distal end of the receiving chamber 113, the push rod 12 can be twisted to separate apart from the neck portion 124, such that the used syringe needle 14, including the seat 13 and a front segment of the push rod 12 are kept in the syringe cylinder 11 to prevent injuring medicinal personals.

While we have shown and described various embodiments in accordance with the present invention, it is clear to those skilled in the art that further embodiments may be made without departing from the scope of the present invention.

What is claimed is:

1. A safety syringe with a needle retraction device comprising:
a syringe cylinder having a receiving chamber to receive medicinal liquid, a push rod inserted in the receiving chamber of the syringe cylinder to push the medicinal liquid forward, a seat disposed at a front end of the syringe cylinder to receive a syringe needle, and a protective sleeve for covering the seat and the syringe needle, wherein
the push rod includes a hollow and raised pushing knob with a predetermined diameter mounted at a top end thereof, the pushing knob includes a stem extending downward from a bottom thereof, the stem includes a combining portion fitted thereon and an engaging rim fixed at a lower side thereof to prevent the medicinal liquid from leak, the engaging rim includes a neck portion mounted at a lower side thereof and having a reduced diameter, and a lower side of the neck portion is connected with a plurality of longitudinal ribs;
the seat includes a fitting post with a reduced diameter fixed at a front end thereof to project out of the syringe cylinder, the fitting post includes a passage formed at a central portion thereof to receive the syringe needle, and the passage includes a connection unit provided at a rear end thereof, at an opening end of the connection unit is mounted a first diametered segment, a diameter of which is larger than an outer diameter of the pushing knob of the push rod, and a second diametered segment with a smaller diameter is formed in the connection unit to communicate with the passage, between the first and the second segments is defined with a reduced segment so that the pushing knob is guided by the first diametered segment to be moved into the connection unit, and the pushing knob is compressed to reduce its outer diameter so as to be forced into the second diametered segment, and then the pushing knob returns its original size to be positioned in the reduced segment;
wherein the pushing knob includes a through bore arranged therein and communicating with the receiving chamber, the through bore is composed of a laterally-oriented perforation and two openings disposed at both ends of the perforation.

2. The safety syringe with a needle retraction device as claimed in claim 1, wherein the pushing knob of the push rod is inclined at a predetermined angle in relation to the stem.

3. The safety syringe with a needle retraction device as claimed in claim 1, wherein the stem includes a combining portion fitted thereon and having a first protrusion and a second protrusion, diameters of the first and the second protrusions are in response to an inner diameter of the receiving chamber of the syringe cylinder, and the combining portion further has a groove defined between the first and the second protrusions and thereof.

4. The safety syringe with a needle retraction device as claimed in claim 1, wherein the connection unit of the seat includes a slot arranged at an outer surface thereof to receive an anti-leak ring.

5. The safety syringe with a needle retraction device as claimed in claim 1, wherein at a suitable position of the fitting, post is engaged a peripheral projection.

6. The safety syringe with a needle retraction device as claimed in claim 1, wherein at a lower side of the fitting post is disposed a positioning portion which is biased against a stop portion, and the stop portion is defined between a receiving hole of the syringe needle of the syringe cylinder and an interior of the receiving chamber.

7. The safety syringe with a needle retraction device as claimed in claim 1, wherein the through bore includes an upper diameter which is gradually enlarged toward the pushing knob and includes a lower diameter which is gradually reduced toward the combining portion.

8. A safety syringe with a needle retraction device comprising:
a syringe cylinder, a push rod inserted in the syringe cylinder, a seat disposed at a front end of the syringe cylinder to receive a syringe needle, wherein the push rod includes a pushing knob with a through bore mounted at a top end thereof; the through bore is composed of a laterally-oriented perforation and two openings disposed at both ends of the perforation; and the seat includes a connection unit provided at a rear end thereof, the pushing knob allows to be pushed into the connection unit, because the pushing knob is pressed to reduce its outer diameter by using the through bore.
